(19) United States
(12) Patent Application Publication
(10) Pub. No.: US 2022/0174902 A1
(43) Pub. Date: Jun. 9, 2022

(54) In-Vitro Photoautotrophic Propagation of Cannabis

(71) Applicant: Node Labs Inc., Petaluma, CA (US)

(72) Inventor: Christopher Leavitt, Petaluma, CA (US)

(21) Appl. No.: 17/677,719

(22) Filed: Feb. 22, 2022

Related U.S. Application Data
(63) Continuation of application No. PCT/US20/46645, filed on Aug. 17, 2020.
(60) Provisional application No. 62/888,853, filed on Aug. 19, 2019.

(51) Int. Cl. A01H 4/00; A01H 5/12; A01H 6/28; A01G 2/10; A01G 22/00

(52) U.S. Cl. CPC A01H 4/005; A01H 5/12; A01G 22/00; A01G 2/10; A01H 6/28

(57) ABSTRACT

A plant propagation system, process and method are provided for promoting the growth of plant tissue into propagules using a photoautotrophic gel system. The plant propagation system includes a sterile growth vessel that has a vented lid to permit passive diffusion of gases. The process is initiated with one or more sterile rooted explants, which are then cultured in a large container with a vented lid photoautotrophically, which simulates ex-vitro growth conditions. These nodal explants can then be rooted onto photoautotrophic rooting agar gel in vented lid containers and subsequently transferred onto a substrate of choice for mature growth ex-vitro.

### CROSS REFERENCE

This application is a Continuation application of International Patent Application No. PCT/US20/46645 filed Aug. 17, 2020, which claims priority to U.S. Provisional Application No. 62/888,853 filed Aug. 19, 2019, the contents of which are herein incorporated by reference in their entirety.

### FIELD ACCORDING TO THE INVENTION

The invention generally relates to aseptic in-vitro propagation of plant tissue under photoautotrophic conditions.

### BACKGROUND

Cannabis is composed of three species of which there are numerous hybrids: Cannabis indica, Cannabis sativa, and Cannabis ruderalis. Cannabis sativa L. is commonly used to denote the domesticated cannabis associated with agronomic use. Its agronomic value is split between varieties that contain psychoactive or therapeutic chemicals such as Cannabidiol (CBD) and Tetrahydrocannabinol (THC), and hemp varieties, which are valued primarily for their fibers and other products. Cannabis is a dioecious plant, having male and female reproductive organs in separate plants; although only female plants produce flowers that contain enough valuable cannabinoids to render them commercially desirable. Breeding of cannabis has introduced many unique varieties, which can only be reproduced by means of asexual reproduction. Therefore, to ensure that all resulting plants are both female and true-to-type, asexual cloning is traditionally used as the preferred method of propagation.

Ex-vitro cloning or conventional cloning refers to a process in which 4-6-inch-long stem sections with a few leaves of a Cannabis plant are dissected and dipped into a rooting hormone such as IBA (indole-3 butyric acid), and placed into a moist substrate such as soil, peat, or rockwool. Under suitable humidity conditions these cut stems develop roots and become miniature plants. However, this process has drawbacks. The primary drawback is that the propagated plant shares that same diseases, insect infestations, and/or viruses as its parent.

In addition, repeated manipulation of the mother plant exposes it to pathogens which can infect the cut wounds. Fusarium and Pythium are two plant pathogens that are very common and are known to infect stressed clone mothers, which systematically infect the clones it produces. The cloning process gets severely compromised when the clone mother plants become severely infected and resulting endogenous contamination can impact yield and quality of the crop it is meant to produce. Overall, clone nurseries struggle with long-term plant pathogen prevention. This problem is not unique to Cannabis cultivation and similar issues have been reported in cultivation of plants such as avocados, bananas, orchids soybeans, and berries. Often, nurseries resort to the use of heavy doses of antibiotics and fungicides to reduce infection, which in turn increases costs, impacts the environment and lowers the quality of the propagule making it less attractive for organic growers.

Micropropagation is a method to produce genetically identical plantlets using aseptic tissue culture techniques. Carbon dioxide concentration, photosynthetic photon flux, relative humidity, and air flow in the growth vessel are some of the most important environmental factors affecting plantlet growth and development; controlling these factors requires knowledge and techniques of greenhouse and horticultural engineering as well as the knowledge of physiology of in-vitro plantlets. Micropropagation allows rapid propagation of uniform plantlets of strains that are free of diseases and pests.

The genetic progress of any Cannabis breeding programs is limited due to the difficulty in maintaining selected genotypes under field or greenhouse conditions because of the allogamous (cross-fertilization) nature of species. It is therefore impossible difficult to maintain elite cultivar/clones by seed. Storage of seed, which can be highly variable and produce less efficacious genotypes (Meijer et al. 1992; Meijer E. P. M.; van dr Kamp H. J.; van Eeuwijk F. A. Characterisation of Cannabis accessions with regard to cannabinoid content in relation plant characters. Euphytica 62: 187-200;1992.). Plant regeneration protocols have been developed for different Cannabis genotypes and explant sources (Loh et al. 1983; Loh W. H. T.; Hartsel S. C.; Robertson W. Tissue culture of Cannabis sativa L. and in vitro biotransformation of phenolics. Z. Pflanzenphsiol 111: 395-400; 1983, Richez-Dumanois et al. 1986; Richez-Dumanois C.; Braut-Boucher F.; Cosson L.; Paris M. Multiplication vegetative in vitro du chanvre (Cannabis sativa L.) application a la conservation des clones selectionnes. Agronomie 6: 487-495; 1986. Mandolino and Ranalli 1999, Mandolino G.; Ranalli P. Advances in biotechnological approaches for hemp breeding and industry. In: Ranalli P. (ed) Advances in hemp research. Haworth, N.Y., pp 185-208; 1999.; Slusarkiewicz-Jarzina et al. 2005; Slusarkiewicz-Jarzina A.; Ponitka A.; Kaczmarek Z. Influence of cultivar, explant source and plant growth regulator on callus induction and plant regeneration of Cannabis sativa L. Acta Biol. Crac. Ser. Bot. 472: 145-151; 2005, Bing et al. 2007, Bing X.; Ning, L.; Jinfeng T.; Nan G. Rapid tissue culture method of Cannabis sativa for industrial uses. CN 1887043 A 20070103 Patent (p. 9); 2007.), and considerable variation has been reported in the response of cultures and in the morphogenic pathway. A report by Fisse et al. (Fisse J.; Braut F.; Cosson L.; Paris M. Etude in vitro des capacities organogenetiques de tissues de Cannabis sativa L. Effet de differentes substances de croissance. Planta Med. 15: 217-223; 1981.) assessed organo-genesis but did not observe any direct organ formation on explants and reported that Cannabis calluses readily produced roots but were unreceptive to shoot formation. Mandolino and Ranalli (Mandolino G.; Ranalli P. Advances in biotechnological approaches for hemp breeding and industry. In: Ranalli P. (ed) Advances in hemp research. Haworth, N.Y., pp 185-208; 1999) reported occasional shoot regeneration from calluses. Feeney and Punja (Feeney M.; Punja Z. K. Tissue culture and agrobacterium-mediated transformation of hemp (Cannabis sativa L.). In Vitro Cell. Dev.Biol. Plant 39: 578-585; 2003.) failed to regenerate hemp plantlets, either directly or indirectly from callus or suspension cultures.

Plant survival, growth, and productivity are intimately coupled with the aerial environment through processes such as energy exchange, loss of water vapor in transpiration, and uptake of carbon dioxide in photosynthesis (Stoutjesdijk and Barkman 1992; Stoutjesdijk P. H.; Barkman J. J. Microclimate, vegetation and fauna. Opulus, Sweden; 1992). The water vapor exchange rate affects the energy budget and transpiration of leaves and, consequently, the physiology of the whole plant (Chandra and Dhyani 1997; Chandra S.; Dhyani P. P. Diurnal and monthly variation in leaf temperature, water vapour transfer and energy exchange in the leaves of Ficus glomerata during summer. Physiol. Mol. Biol. Plants 3: 135-147; 1997).

Many micropropagation protocols for growing Cannabis strains have been published (Casano S, Grassi G (2009) Valutazione di terreni di coltura per la propagazione in vitro della canapa (Cannabis sativa). Italus Hortus 16:109-112; Lata H, Chandra S, Khan I A, ElSohly M A (2016a) In vitro propagation of Cannabis sativa L. and evaluation of regenerated plants for genetic fidelity and cannabinoids content for quality assurance. Methods in molecular biology. Springer, Clifton, pp 275-28). However, many of these protocols are difficult to reproduce, and many strains are intolerant of or display negative symptoms during heterotrophic tissue culture such as hyperhydricity, chlorosis, necrosis, and excessive callusing. The instant invention aims to provide a reliable and productive micropropagation method that yields vigorous plantlets with desired genetic characteristics identical to the stock or the clone mother plant using a completely photoautotrophic multiplication cycle.

### SUMMARY OF THE INVENTION

The invention aims to provide systems, methods and devices for the aseptic in-vitro propagation of plant tissues under photoautotrophic conditions for producing high quality clone mothers and propagules that are free of pathogenic contamination.

The invention provides a method of in vitro propagation of plant tissue, said method comprising:

a. placing a first node segment of a plant in a low sugar gel media contained in a semi permeable vessel,

b. exposing the vessel to a light environment,

c. permitting passive diffusion of gases from the vessel for a time sufficient to facilitate photosynthetic growth of the first node segment, said photosynthetic growth resulting in the propagation of the first node segment into a propagule comprising one or more node segments.

In certain embodiments of the invention, the low sugar gel media comprises less than 5 g of soluble carbohydrates, the soluble carbohydrates are selected from the group consisting of sucrose, glucose, fructose, galactose, and maltose. In some embodiments, the low sugar gel media contains a cytokinin and/or an auxin.

In certain embodiments of the invention the light environment is a low light environment. In some embodiments, the light environment has a flux of less than 100 μmol/m−2/s−1. In some embodiments, the low light environment is less than 30 μmol/m−2/s−1.

In certain embodiments of the invention the time sufficient is 10-40 days. In some embodiments. In some embodiments, the time sufficient is 10-40 days. In other embodiments, the time sufficient is 7-10 days.

In certain embodiments of the invention, the first node segment is at least 1 cm in length. In some embodiments, the first node segment is obtained by dissecting stem sections of a sterile clone mother plant. In some embodiments, the method further comprises the step of dissecting said one or more node segments to generate a plurality of node segments and repeating steps (a) to (c).

In some embodiments of the invention, the vessel comprises a vented lid which permits said passive diffusion of gases for photosynthetic growth. In some embodiments, the vented lid comprises a pore size of 0.2 microns.

In certain embodiments, the plant is selected from the group consisting of Cannabis sativa, Cannabis indica, and Cannabis ruderalis. In certain embodiments the propagule comprises one or more fibrous roots.

The invention provides a method of in vitro multiplication of plant tissue, said method comprising:

a. placing a propagule in a solid substrate or aggregate augmented with a liquid nutrient solution contained in a semi permeable vessel,

b. exposing the vessel to a light environment,

c. permitting a passive diffusion of gases from the vessel for a time sufficient to facilitate photosynthetic growth of the first node segment, said photosynthetic growth resulting in the propagation of the first node segment into one or more node segments.

In certain embodiments, the light environment is a moderate light environment. In some embodiments, the moderate light environment has a flux of less than 66 μmol/m⁻²/s⁻¹.

In certain embodiments of the invention the time sufficient is 10-40 days. In some embodiments. In some embodiments, the time sufficient is 10-40 days. In other embodiments, the time sufficient is 7-10 days.

In certain embodiments, the growth vessel further comprises a lid. In some embodiments, the lid is a vented lid. In some embodiments, the vented lid prevents the entry of pathogenic microbes into the growth vessel. In some embodiments, the vented lid facilitates gaseous exchange and water vapor between the vessel and its surrounding environment. In some embodiments, the vented lid comprises spun bound or non-woven polypropylene membrane or fabric. In some embodiments, the fabric has a pore size ranging from 0.01-0.2 microns.

The invention also provides a lid for attachment to a growth vessel having an open top, wherein said growth vessel is suitable for photoautrophic plant growth and said lid configured to seal said open top of said growth vessel, wherein said lid comprises an aperture covered by a membrane capable of permitting free exchange of gases and water vapor into and out of the growth vessel, and wherein said membrane prevents the entry of microbial pathogens into said growth vessel. In some embodiments, the size of aperture ranges from 2.5 cm to 7 cm. In some embodiments, the membrane has a pore size ranging from 0.01-0.2 microns. In some embodiments, the diameter of lid ranges from 5 to 50 cm. In some embodiments, the lid is made from materials selected from the group consisting of polypropylene, polyethylene, poly ethylene terephthalate, poly vinyl chloride, polycarbonate, polystyrene, and glass. In some embodiments, the membrane is made from material selected from polypropylene, cellulose, and nylon.

In one aspect, the invention provides a method of in vitro propagation of plant tissue, said method comprising: a.placing a first node segment of a plant in a low sugar gel media contained in a semi permeable vessel, b. exposing the vessel to a light environment, c. permitting passive diffusion of gases from the vessel for a time sufficient to facilitate photosynthetic growth of the first node segment, said photosynthetic growth resulting in the propagation of the first node segment into a propagule comprising one or more node segments. In another aspect, the low sugar gel media comprises less than 5 g of soluble carbohydrates, the soluble carbohydrates are selected from the group consisting of sucrose, glucose, fructose, galactose, and maltose. In another aspect, the low sugar gel media contains a cytokinin and/or an auxin. In another aspect, the light environment is a low light environment. In some aspects, the light environment has a flux of less than 100 μmol/m−2/s−1. In some aspects, the low light environment is less than 30 μmol/m−2/s−1. In another aspect, the time sufficient is 10-40 days. In another aspect, the time sufficient is 10-40 days. In other aspects, the time sufficient is 7-10 days. In another aspect, the first node segment is at least 1 cm in length. In another aspect, the first node segment is obtained by dissecting stem sections of a sterile clone mother plant. In another aspect, the method further comprises the step of dissecting said one or more node segments to generate a plurality of node segments and repeating steps (a) to (c). In another aspect, the vessel comprises a vented lid which permits said passive diffusion of gases for photosynthetic growth. In another aspect, the vented lid comprises a pore size of 0.2 microns. In another aspect, the plant is selected from the group consisting of Cannabis sativa, Cannabis indica, and Cannabis ruderalis. In another aspect, the propagule comprises one or more fibrous roots. In one aspect, the invention provides a method of in vitro multiplication of plant tissue, said method comprising: a. placing a propagule in a solid substrate or aggregate augmented with a liquid nutrient solution contained in a semi permeable vessel, b. exposing the vessel to a light environment, c. permitting a passive diffusion of gases from the vessel for a time sufficient to facilitate photosynthetic growth of the first node segment, said photosynthetic growth resulting in the propagation of the first node segment into one or more node segments. In another aspect, the light environment is a moderate light environment. In another aspect, the moderate light environment has a flux of less than 66μmol/m⁻²/s⁻¹. In another aspect, the time sufficient is 10-40 days. In another aspect, the time sufficient is 10-40 days. In other embodiments, the time sufficient is 7-10 days. In another aspect, the growth vessel further comprises a lid. In one aspect, the lid is a vented lid. In one aspect, the vented lid prevents the entry of pathogenic microbes into the growth vessel. In one aspect, the vented lid facilitates gaseous exchange and water vapor between the vessel and its surrounding environment. In one aspect, the vented lid comprises spun bound or non-woven polypropylene membrane or fabric. In another aspect, the fabric has a pore size ranging from 0.01-0.2 microns.

### BRIEF DESCRIPTION OF THE FIGURES

FIG. 1 shows schematic representation of growth processes and its various phases.

FIG. 2 shows a photograph of a plant in the acclimatization stage of the plant propagation process grown under heterotrophic conditions.

FIG. 3A shows a photograph plants in the root induction stage of the plant propagation process grown under photoautotrophic conditions.

FIG. 3B shows a photograph of the underside of plants in the root induction stage of the plant propagation process grown under photoautotrophic conditions.

FIG. 4 shows a photograph of plants in the acclimatization stage of the plant propagation process grown under photoautotrophic conditions.

FIG. 5 shows a photograph of a large sterile clone mother with healthy turgid leaves grown aseptically under photoautotrophic conditions.

FIG. 6 shows photographic examples of a vented lid containing a filter of 0.2-micron pore size.

FIG. 7 shows a photograph indicating the comparison of plants grown under photoautotrophic conditions in vented and sealed growth chambers.

FIGS. 8A-8D show the process of production of vented lids. FIG. 8A shows a representative example of the plastic lid before a ventilation orifice is created. FIG. 8B shows the gas and moisture permeable membrane made from spun bound or non-woven polypropylene fabric. FIG. 8C shows the production of an aperture in the lid and permeable membrane that will be used to cover the aperture. FIG. 8D shows the aperture of the lid is affixed with gas and moisture permeable membrane by means of adhesive resulting in the production of a representative example of the vented lid for growth vessel.

FIG. 9 shows a vented lid wherein the gas and moisture permeable membrane is affixed to the aperture of the lid using an ultrasonic welder.

### DETAILED DESCRIPTION OF THE INVENTION

It is to be understood by those of ordinary skill in the art that the present discussion is a description of exemplary embodiments only and is not intended as limiting the broader aspects of the present invention.

The present invention generally pertains to a plant propagation system, process and devices for promoting the growth of plant tissue into propagules. The plant propagation system includes a sterile growth vessel that has a vented lid to permit passive diffusion of gases. The process is initiated with one or more sterile rooted explants, which are then cultured in a large container with a vented lid photoautotrophically, which simulates ex-vitro growth conditions. These nodal explants can then be rooted onto photoautotrophic rooting agar gel in vented lid containers and subsequently transferred onto a substrate of choice for mature growth ex-vitro.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, illustrative methods and materials are described. As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As defined herein, the term “heterotrophic propagation” refers to a process wherein an in-vitro plant derives greater than 20% or all of its energy from a carbohydrate source such as sucrose, which is delivered directly through the gel or liquid media.

As defined herein, the term “photoautotrophic propagation” refers to a process wherein an in-vitro plant derives greater than 80% or all of its energy from photosynthesis.

As defined herein, the term “photosynthetic growth”: refers to the growth of one or more leaves from a cut plant section that is exposed to nutrients and a suitable light environment. The light conditions can vary having a flux ranging from 30-100 μmol/m⁻²/s⁻¹ and the nutrients may be provided in the form of a sterile low-strength liquid hydroponics media.

As defined herein, the term “time sufficient” refers to the time period that a plant takes to achieve the desired photosynthetic growth—generally the time period is between 7-30 days, preferably 10-21 days, more preferably 10-14 days.

As defined herein, the term “clone mother” or “mother stock” refers to an exemplary plant from a single chain chosen for its unique phenotype such as ratio of concentration between Tetrahydrocannabinol (THC) and Cannabidiol (CBD), color and shape of leaves, aroma, resin production, flowering cycles, height, etc. Clone mothers are generally grown to preserve the root stock and to generate propagules with identical desired traits to facilitate commercial propagation. Typically clone mothers are grown to height of 5-25 centimeters and have at least five leaf nodes. Once a desired height and desired number of leaf nodes are reached, the clone mothers can then be used for generating cut sections which later grow into propagules.

As defined herein, the term “in vitro clone mother” refers to a clone mother plant (as defined above) grown in a sterile environment that is grown generating propagules therefrom.

As defined herein, the term “meristem” refers to a microscopic cluster of plant cells consisting of apical meristem tissue weighing in the range of 0.1 and 2 milligrams and with a length of no more than 1 millimeter.

As defined herein, the term “shoot tip” refers to a dissection taken from a plant consisting of a bud containing a somatic meristem weighing in the range of 1 and 5 milligrams and with a length in the range of 0.5-5 millimeters.

As defined herein, the term “flux” refers to a unit of measure light.

As defined herein, the term “node or node segment” refers to a plant part which is a stem section having a length in the range of 1 to 5 centimeters, inclusive, and a bud containing a somatic meristem that may or may not include a leaf and petiole.

As defined herein, the term “gel” refers to a solution suspension using a gelling agent such as agarose, gellan gum, or gelatin that maintains shape as a solid.

As defined herein, the term “acclimatization” refers to the process of transitioning an in-vitro plant to tolerate higher light levels, air movement, and nutrient concentration associated with growth outside of a vessel and in the outside world. Acclimatization, as used here, may occur over a time period of 24-48 hours, 10-30 days, and/or 1-4 weeks, depending on the nutrients, air gas contents and movement, and light exposure to which the plant is exposed over time.

As defined herein, the term “sponge cube” refers to an open cell foam derived from polyurethane or other polymers with a volume of 1-500 cubic centimeters.

As defined herein, the term “propagule” or “plantlet” refers to a dissection taken from a larger plant that will yield a new plant upon maturation. “Plantlet” as used herein refers to a young or small plant with roots and shoots. In some embodiments of the present invention, the plantlets are 0.5-10 centimeters in height. “Propagule” as used herein refers to plants and plantlets that are used for propagating a plant and are ready to be transplanted into soil or other planting medium with a length of 0.5-10 centimeters.

As defined herein, the term “somaclonal variation” is a genetic variation observed among progeny of plants regenerated from somatic cells cultured in vitro.

As defined herein, the term “low sugar media” refers to a growth media that comprises less than 5 grams of soluble carbohydrates, in some embodiments, the soluble carbohydrate is sucrose. A low sugar gel media refers to a growth media that further comprises agarose along with less than 5 grams of soluble carbohydrates.

As defined herein, the term “high sugar media” refers to a growth media that comprises at least 15 grams of soluble carbohydrates, in some embodiments, the soluble carbohydrate is sucrose. A high sugar gel media refers to a growth media that further comprises agarose along with at least 15 grams of soluble carbohydrates.

As defined herein, the term “moderate sugar media” refers to a growth media that comprises between 5 and 15 grams of soluble carbohydrates, in some embodiments, the soluble carbohydrate is sucrose. A moderate sugar gel media refers to a growth media that further comprises agarose along with between 5 and 15 grams of soluble carbohydrates.

As defined herein, the term “low light environment” refers to a light environment that has a flux of less than 30 μmol/m⁻²/s⁻¹.

As defined herein, the term “high light environment” refers to refers to a light environment that has a flux of greater than 100 μmol/m⁻²/s⁻¹.

As defined herein the term “moderate light environment” refers to refers to a light environment that has a flux of between 30 and 100 μmol/m⁻²/s⁻¹.

As defined herein, the term “sterile” (such as “sterile clone mother” or “sterile plant”) refers to plants, propagules and cut sections that are free from pathogenic contamination such as bacteria, fungi and viruses. An infection caused by one or more pathogenic organisms such as bacteria, fungi or viruses can be determined by visual inspection of the plant and the media in which its grown. Common symptoms of plant infection may include formation of powdery mildew, yellow or brown leaf spots, and colonization of the media by fungal or bacterial colonies.

As defined herein the term “sterile growth medium” refers to commonly used sterile growth media such as sponge cubes, rock wool, vermiculite, perlite or clay pellets that are free from pathogenic organisms.

As defined herein the term “sterile propagules” refers to propagules that are free from pathogenic contamination and are often generated from a sterile clone mother plant.

As defined herein the term “auxin” refers to any compound with auxin or auxin-like activity. “Auxin-like activity” refers to the typical activity observed in a plant as a result of treatment with auxin. Thus, a compound with auxin-like activity promotes the formation of unorganized cell mass on explants at high concentration, promotes apical dominance, maintains cell proliferation alone or in the presence of cytokinin and/or induces root development on shoot cuttings.

As defined herein the term “cytokinin” refers to any compound with cytokinin or cytokinin-like activity. “Cytokinin-like activity” refers to the typical activity observed in a plant as a result of treatment with cytokinin. Thus, a compound with cytokinin-like activity promotes shoot regeneration from cell cultures, promotes auxiliary shoot growth and/or maintains cell proliferation in the presence of auxin.

As defined herein, the term “increased tolerance to an environmental condition” means the ability of a plant to withstand a deleterious environmental condition that would normally be harmful or deleterious to a wild-type plant of the same species.

As used herein, the term “subculture,” or “passage” refers to the transfer of cells from one culture vessel to another; this usually involves the subdivision of a proliferating cell culture. Thus, “subculture” is the process by which the tissue or explant is subdivided and then transferred into fresh culture medium.

As used herein, the term “media” useful for the primary cultivation step (i.e., the primary medium) can be any basal medium used for plant tissue culture. Such media are well known to those of skill in the art. In one embodiment, the primary medium is supplemented/augmented with at least one plant hormone (i.e., plant growth regulator). Examples of suitable plant hormones include auxins and cytokinins. Auxins include any compound with auxin-like activity. Thus, auxins may include, but are not limited to, 2,4-dichlorophenoxyacetic acid, picloram, and indolebutyric acid (IBA) and combinations thereof. Cytokinins include also any compound with cytokinin-like activity. Thus, cytokinins may include, but are not limited to, thidiazuron, zeatin, benzyladenine, kinetin, adenine hemi sulfate and dimethylallyladenine, and combinations thereof. In some embodiments, the primary medium is supplemented with at least one auxin and at least one cytokinin.

As used herein the term “soluble carbohydrates” refers to carbon sources that are known to those of ordinary skill in the art (Slater et al. Plant Biotechnology, the Genetic Manipulation of Plants, Oxford University Press, 368 pp., (2003)) and include, but are not limited to, sugars such as sucrose, fructose, fucose, glucose, maltose, galactose and sorbitol, and the like.

As used herein the term “one or more node segments” refers to the formation of a stem section containing one or more buds containing a somatic meristem that may include a leaf and petiole.

As used herein the term “solid substrate” refers to any sterile and inert grow media which is not a liquid in itself but has the ability wherein nutrient solutions can be optionally infused, common examples of solid substrates include urethane, rock wool, sponge cube substrates, coco coir, perlilte, vermiculite, volcanic lava rocks and also a mixture of blond and black peat, fibrous peat, tree bark, perlite and worm humus can be used.

As used herein the term “solid aggregate substrate” refers to a compound mixture of one or more grow media such as clay pellets, rock wool, sponge cube substrates, coco coir, perlite, vermiculite, volcanic lava rocks, peat, fibrous peat, tree bark, perlite and worm humus.

As used herein the term “non-gel solid substrate” refers to any grow media that does not contain agarose in its composition.

As used herein the term “vessel” refers to a small, enclosed and portable container used for growing plants. It may take the form of a pot, square or rectangular box, tub, or flask. Generally, these grow vessel are made from plastic such as high-density polyethylene (HDPE) or low-density polyethylene (LDPE) or polypropylene (PP) or polyethylene terephthalate (PET). In some embodiments, the grow vessels can be made from glass or terracotta. The vessel can range in size from 0.2 liter (8 ounces) to 15 liters (550 ounces) depending upon the phase of the growth process, the size and the number of plants being grown in it.

As used herein the term “semi permeable vessel” refers to a grow vessel fitted with a lid that is permeable to exchange of gases but impermeable to the entry of fungus, bacteria or viruses.

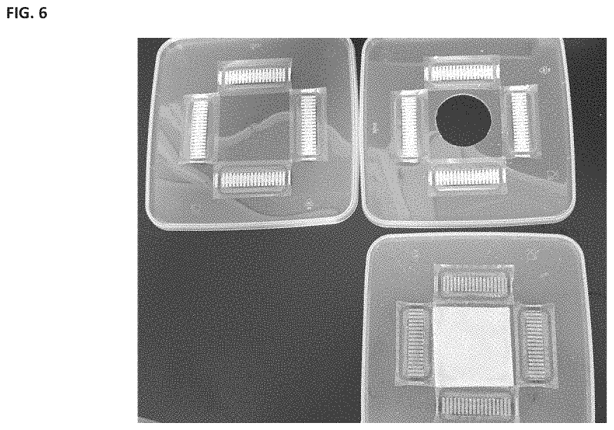

As used herein the term “vented lid” refers to a lid that covers the grow vessel and contains an opening that is covered with a filter paper. The smaller pore size of the filter paper renders the grow vessel semi permeable as it facilitates passive exchange of gases in and out of the grow vessel but blocks the entry of pathogenic organisms and spores. Generally, the pore size of the filter paper in the vented lid ranges from 0.01 to 0.2 microns. An example of vented lid is show in FIG. 6.

As used herein the term “high success rate” refers to the percentage of plant sections that have developed roots during the root induction phase of the grow process. Generally, the rooting plant of sections is considered the most difficult stage in traditional propagation and on an average only 40-50% of cut sections develop roots. If more than 90% of the cut sections develop one or more fibrous roots during the root induction phase of the grow process, then the growth process is deemed to have a high success rate.

As used herein, the term “ultrasonic welding” refers to industrial technique whereby high-frequency ultrasonic acoustic vibrations are locally applied to workpieces being held together under pressure to create a solid-state weld. It is commonly used for plastics and metals, and especially for joining dissimilar materials. In ultrasonic welding, there are no connective bolts, nails, soldering materials, or adhesives necessary to bind the materials together. Ultrasonic welders are devices that are used for producing an ultrasonic weld, common examples include hand held welders from Sonitek™.

As used herein, the term “hyperhydricity” refers to a physiological malformation that results in excessive hydration, low lignification, impaired stomatal function and reduced mechanical strength of tissue culture-generated plants. Main causes of hyperhydricity in plant tissue culture include oxidative stresses, high salt concentration, high relative humidity, low light intensity, gas accumulation in the atmosphere of the jar, and length of time intervals between subcultures.

As used herein, the term “chlorosis” refers to yellowing of leaf tissue due to a lack of chlorophyll. It results from failure of chlorophyll to develop because of infection by a virus; lack of an essential mineral or oxygen; injury from alkali, fertilizer, air pollution, or cold; insect, mite, or nematode feeding etc. Affected leaves turn yellow, except for the veins, which remain green. In severe cases, foliage may turn brown and die.

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

Ranges: throughout this disclosure, various aspects according to the invention can be presented in a range format. The description in range format is merely for convenience and brevity and should not be construed as an inflexible limitation on the scope according to the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

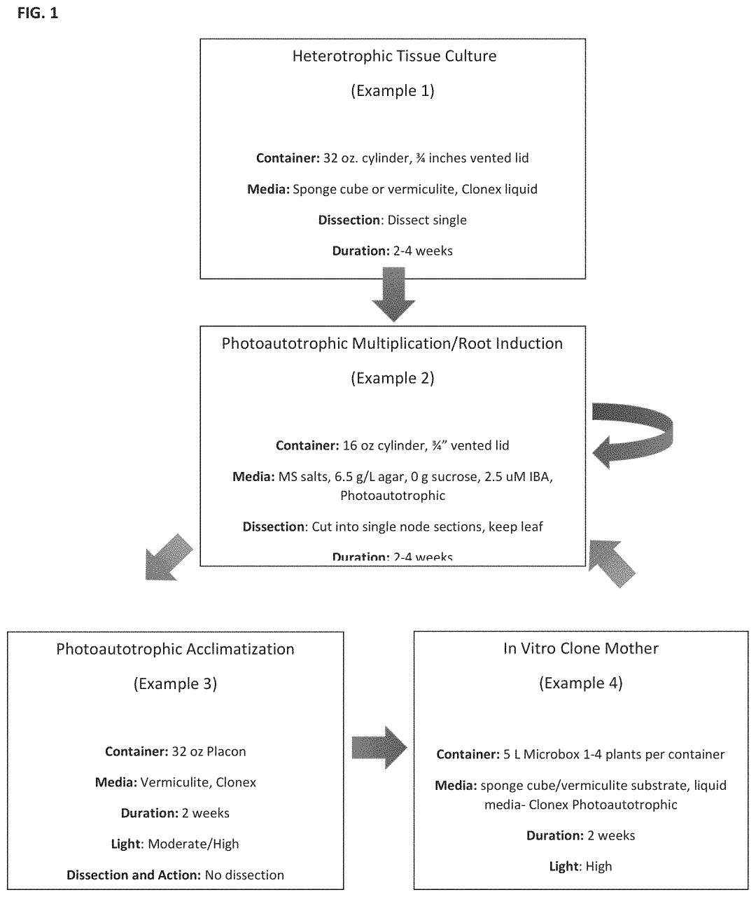

The plant propagation system and method of the present invention may be better understood by reference to the following examples. Each example depicts a single step or a phase in the growth process. An overview of the various phases of the grow process are schematically depicted in FIG. 1. Examples 1 describe the phases of plant tissue culture that is carried out under heterotrophic conditions. Examples 2-4 describe the phases of root induction/multiplication, acclimatization and generation of clone mother that are carried out under photoautotrophic conditions. Phases described in examples 2-4 are collectively referred to as the photoautotrophic micropropagation stage.

In some embodiments of the invention, the steps carried under heterotrophic conditions are optional. In some embodiments of the invention, the growth process comprises one or more steps carried under heterotrophic conditions and one or more steps carried under photoautotrophic conditions. In some embodiments of the invention, the growth process comprises one or more steps under heterotrophic conditions that are repeated several times to ensure multiplication of propagules. In some embodiments of the invention, the growth process comprises one or more steps under photoautotrophic conditions that are repeated several times to ensure multiplication of propagules.

Unless otherwise noted, all growth vessels used in the examples preceding below have been sterilized using gamma radiation. Solid and porous grow media such as vermiculite, sponge substrates and rockwool have been sterilized using specialized vented vessels in an autoclave. All nutrient solutions, buffers and water have also been sterilized using an autoclave and excisions of plant tissues are conducted inside a laminar flow hood to ensure an aseptic environment at all growth phases. All these plants cut sections and plantlets or propagules were kept under similar environmental conditions grown various containers on racks with two 13 watt LED lights (Hort Americas, Tex.) per shelf. Using an automatic electric timer, artificial day/night cycle was regulated with a 18-h photoperiod. Grow room temperature and relative humidity was kept nearly 25-35° C. and 60%, respectively. In some embodiments, the temperature was maintained at 30° C. to promote optimal growth. To determine the effect of light on growth process, leaves were exposed to different photosynthetic photon flux densities (PPFD) viz., 0, 5, 10, 15, 20, 25, 30, 35, 40, 50, 55, 60, 70, 80, 90 and 100 μmol/m⁻²/s⁻¹ with the help of an artificial light source (Model LI-6400-02; light emitting silicon diode; LI-COR).

### Example 1: Heterotrophic Tissue Culture

The first phase of the propagation process is to procure plantlets from traditional tissue culture. This process is comprised of four steps: initiation, elongation/multiplication, rooting, and acclimatization. This process is described by Lata et al., 2009. Using sanitized shears, node segments or branches are procured from a healthy mother plant with desirable traits. In some embodiments the mother plant is selected on the basis of its chemical profile using gas chromatography—mass spectrometry) high-yielding C. sativa variety grown in an indoor cultivation facility. In some embodiments, the mother plant is provided by the farmers interested in propagating the strain of interest. In some embodiments, the mother plant is procured from a plant dispensary such as Harborside, Oakland, Calif.

The cut sections are washed in an oxidizing solution such as bleach and a detergent solution to disinfest and remove all fungus, insects and bacterial contamination. The cut sections are placed into a jar comprising 15% bleach and 0.1% Tween 20 solution. The jar is shaken for fifteen minutes using a shaker to ensure all cut sections are thoroughly disinfected. The cut sections are further washed in sterile distilled water and the washing is repeated three times to remove all traces of oxidizing solution and detergents. In some embodiments, the cut sections were surface-disinfected using 0.5% NaOCl (15% v/v bleach) and 0.1% Tween 20 for 20 min. The sections were washed in sterile distilled water three times for 5 min prior to inoculation onto the media (Lata et. Al. 2009, Thidiazuron-induced high frequency direct shoot organogenesis of Cannabis sativa L., In Vitro Cell.Dev.Biol.-Plant, 45:12-19).

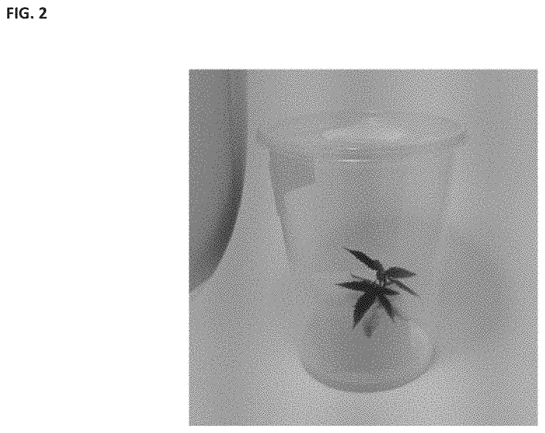

The sterile cut sections are then placed onto a sterile surface inside a laminar flow hood to ensure aseptic environment. Using sterile forceps and a scalpel, the cut sections are further delineated into node segments, shoot tips or meristem explants. The explants are then placed in a growth vessel comprising a gel media that contains a high sucrose content, an auxin and a cytokinin. In some embodiments the gel media comprises (Murashige and Skoog's medium; Murashige T.; Skoog F. A revised medium for rapid growth and bioassays with tobacco tissue cultures. Physiol Plant 15: 473-497; 1962.) 3% (w/v) sucrose, 0.8% (w/v) type E agar (Sigma Chemical Co., St. Louis, Mo.) supplemented with various concentrations of cytokinins—benzyladenine (BA), kinetin (Kn), and thidiazuron (TDZ) or in combination with gibberellic acid adjusted to pH 5.7. The growth vessel as seen in FIG. 2 is then exposed to light conditions with an intensity of 40-60 μmol/m⁻²/s⁻¹for a time period of 10-14 days. During this phase, the plants elongate and optionally, the plants can be multiplied by dissecting and replacing onto the same recipe of gel media.

Once the plantlets have developed, they are placed into a root-induction gel. The plantlets are removed from initiation gel media and placed in a growth vessel comprising a primary root induction gel media. The root induction gel media is made with ½ MS medium supplemented with 500 mg 1-1 activated charcoal and 2.5 μM IBA for a 1-liter batch. The growth vessel is then exposed to light conditions with an intensity of 40-60 μmol/m⁻²/s⁻¹ for a time period of 14-21 days or longer, if required. After 21 days, the cut sections are inspected to see if new fibrous roots have formed, once new roots are formed, the sections are then moved to the acclimatization phase (Lata et. Al. 2009, Thidiazuron-induced high frequency direct shoot organogenesis of Cannabis sativa L., In Vitro Cell.Dev.Biol.-Plant, 45:12-19).

In the acclimatization phase, the rooted plantlets or propagules are removed from the root induction media and placed into any non-gel solid or aggregate substrate such as rockwool or vermiculite and several other nutrient solutions commonly known in art can be used such as Clonex Clone Solution at the recommended concentration for new propagules. The growth vessel as seen in FIG. 2 is then placed under light conditions with a flux of 40-60 μmol/m⁻²/s⁻¹ for 5-10 days and then moved to stronger light of 100-150 μmol/m⁻²/s⁻¹. Movement from these different light levels can be modulated depending on preferred growth morphology of the plant and according to maturity and rapidity of growth. Once desired growth level is obtained, a dissection can be taken from this sterile, mature plant and transitioned into the Photoautotrophic Multiplication/Root Induction Gel Phase (Example 2) for stable micropropagation and easy acclimatization.

The heterotrophic tissue culture protocol described above is necessary to provide a sterile, acclimatized plantlet for photoautotrophic micropropagation, but it still has several problems as a stand-alone process. Overall, the plants in heterotrophic gel media lack normal morphology and vigorous growth, often displaying negative symptoms such as hyperhydricity, excessive callus growth, chlorosis and necrosis. Many plants do not survive the process, and certain strains of Cannabis are completely incompatible with it. Transitioning plantlets to a photoautotrophic cycle provides better plant morphology, 80% rooting success, and 95% acclimatization success, and an extremely stable, indefinite growth cycle.

### Example 2: Photoautotrophic Multiplication/Root Induction Gel

Plants create energy for growth using a two-phase process: photosynthesis and cellular respiration. Photosynthesis occurs in chloroplasts inside the leaf of a plant, converting carbon dioxide and water into sugar using the energy from the sun. Cellular respiration then breaks down the sugars using oxygen into usable energy in the form of Adenosine triphosphate (ATP) with water and carbon dioxide as a by-product. Plant tissue culture achieves rapid growth of plant parts that would usually perish by subverting photosynthesis altogether and delivering sugars directly to the plant itself. Such a process of growth is referred to as heterotrophic tissue culture, since photosynthesis isn't completely turned off but drastically decreased.

Heterotrophic tissue culture recognizes that the overall amount of sugars in the cells of plant in-vitro are a combination of overall photosynthesis and the amount of sugars delivered directly to the plant via the gel media. It stands to reason then that the larger the leaves of the plant in culture and brighter the lights are, the more sugars that are being generated by photosynthesis inside the cell. Many plants can ‘shut off’ photosynthesis in the presence of excess sugar, but it was unexpected to determine that Cannabis plants cannot shut off photosynthesis once their leaves start developing during the growth phase. Once the leaf organs develop, the Cannabis propagule immediately begins to exhibit negative symptoms characteristic of overexposure to sugars in the cell such as hyperhydricity, callus formation, and necrosis.

A conventionally tissue-cultured rooted plant is kept sterile through the acclimatization process using increasingly large vented lid containers until it is completely tolerant of significant air movement, a complete lack of carbohydrate in the media and high light levels. The plant is then transplanted into a large plastic container where it is photoautotrophically cultured, allowing the plant to grow with a more normal plant morphology atypical of the tissue culture process. The plant thus grown is used as a mother plant and node segments are dissected off it to generate propagules that are then placed onto photoautotrophic rooting media. The growth process is a fully photoautotrophic micropropagation system since it utilizes a fully rooted and acclimatized clone mother and roots the cut sections directly on to photoautotrophic media. Without wishing to be bound by theory, it has been hypothesized that any plant that has the inability to ‘shut off’ photosynthetic action in the presence of sugars or has increased sensitivity to the symptoms associated with overexposure to sugars could be propagated using this process

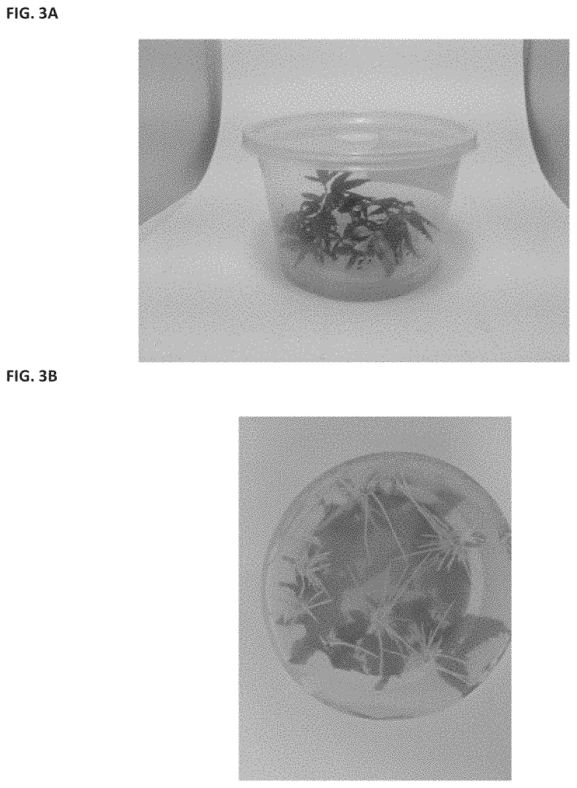

The first step of photoautotrophic propagation process comprises Multiplication/Root Induction phase. A sterile plantlet with a stem measuring at least 1 cm in length and with leaf area of at least 0.5 cm² that has been transitioned away from heterotrophic growth (as in Example 1) and towards photoautotrophic growth is obtained. A stem section is cut from the apical part of the plantlet using sterile forceps and scalpel inside a laminar flow hood. The stem section is cut in such a way that the distal end of the plantlet is left with an intact node and leaf. Node segments from any part of the plantlet can be used during this process including auxiliary shoots and basal nodes segments. The stem section is further dissected into individual node segments of at least 1 cm in length ensuring that there is leaf area of at least 0.5 cm². Sections of multiple nodes can also be used. The nodes are then placed in a growth vessel containing photoautotrophic media. The photoautotrophic media is prepared by dissolving: ½ MS salts, 6.5 g/L agar, 0 to less than 5 g carbohydrate source, preferably sucrose, 1 μg IBA in 1 liter of sterile distilled water. The growth vessel as seen in FIG. 3A and 3B comprises a 0.2-micron vented lid with a diameter of 1.9 cm. Optionally the media can contain an auxin and or a cytokinin. The growth vessel containing nodes embedded in gel media is then placed in light conditions with an intensity of 40-100 μmol/m⁻²/s⁻¹ for a time period of 10-30 days. The nodes are examined after 10-30 days, if the nodes have grown multiple node segments, those newly generated nodes can be dissected and placed onto the same type of gel media in a different container, creating a multiplication cycle. This process is repeated until sufficient quantities of actively growing node sections have been generated.

### Example 3: Photoautotrophic Acclimatization

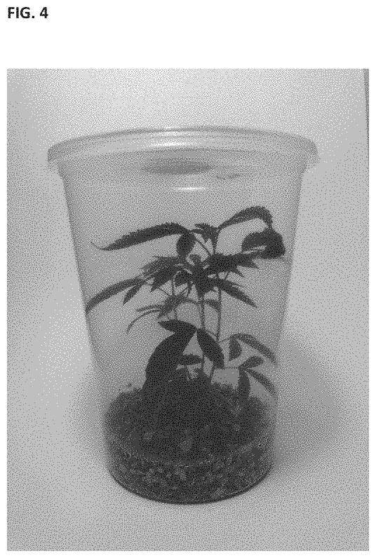

The second step of photoautotrophic propagation process comprises the acclimatization process. The rooted plantlets or propagules are removed from the root induction media of Example 2 and placed into the dibble hole of the sponge cube in a growth vessel containing 150 mL of sterile low-strength liquid hydroponics media. The growth vessel is covered with a 0.2-micron pore size vented lid of a diameter of 3.175 centimeters. The acclimatization process can be performed using any non-gel solid or aggregate substrate such as rockwool or vermiculite and several other nutrient solutions commonly known in art. The growth vessel is as seen in FIG. 4 is then placed under light conditions with a flux of 40-60 μmol/m⁻²/s⁻¹for 5-10 days and then moved to stronger light of 100-150 μmol/m⁻²/s⁻¹. Movement from these different light levels can be modulated depending on preferred growth morphology of the plant and according to maturity and rapidity of growth. Once desired growth level is obtained, the propagule is either packaged and sent for delivery to end customer or it is allowed to grow into a clone mother which can serve as a source for cut sections as in Example 2.

The success of the grow method was determined by evaluating parameters after 30 days of growth such as (a) average number of shoots per explant or cut section, (b) average shoot length, (c) percentage of explants or cut sections producing shoots, (d) average number of roots per explant or cut sections, (e) average root length, and (f) percentage of rooted plantlets. Generally, 1.5 were the average number of shoots observed, 2.5 cm was the average shoot length, 95% was the percentage of cut sections producing shoots, 2 was the average number or roots per cut section, 1 cm was the average root length and 80% was the percentage of rooted plantlets. Rooted plantlets exhibited 95% survival rate 4 weeks after transfer. The acclimatized plants grown by the instant process exhibited normal development and no gross morphological variation was observed.

The grow method and systems disclosed have unique advantages over the existing conventional grow methods. The usage of photoautotrophic propagation in conjunction with heterotrophic propagation allows faster transition of plants from a multiplication round to a fully grown and commercially deliverable product in lesser time. The process also increases overall plant health and reduces the risk of pathogenic contamination which in turn reduces the need for using large concentrations of insecticides, antibiotics and fungicides. The reduction of use in antibiotics, insecticides and fungicides is highly desirable for commercial growers that prefer organic plantlets. The reduction of use of antibiotics also reduces the cost of propagation and lessens the impact to the environment. The process also provides for indefinite storage of clone mothers in boxes which greatly reduces the risk of somaclonal variation which can happen in traditional growth processes. Since clone mothers can be maintained indefinitely, plantlets featuring the same desired traits can be produced without any change in characteristics. The process also allows for reduced use of hormones which can also save costs for the nurseries.

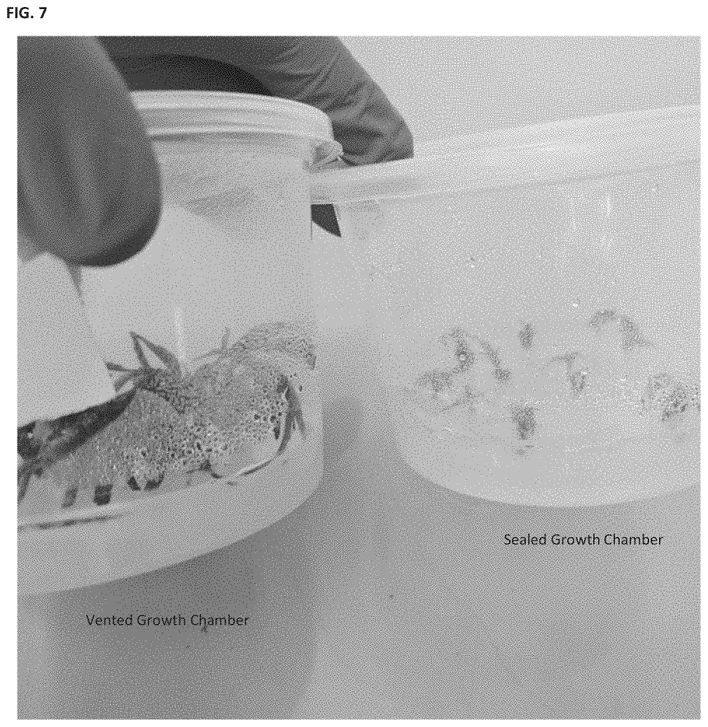

Furthermore, the process has a high success rate in rooting plant sections which is the most difficult stage in traditional propagation. It allows for a larger number of plantlets to be generated and yields of over 25 plantlets from a single in-vitro clone mother have been obtained. It also provides an added advantage of being able to ship the propagules directly to the end customer without needing any further acclimatization since the propagules are grown in full light environments unlike the traditional propagation methods. The process also provides the ability to deliver sterile propagules that are free from contamination and hence the end customer need not go through a sterilization step in their facilities after receiving plants from nurseries. Thus, the aforesaid grow process is superior over the existing methods, as it allows for faster growth, healthier plants, lower costs, lower use of antibiotics, insecticides and fungicides, and ideal propagules for organic commercial plant farms. The experiments when repeated with sealed lids and vented lids, indicated that the vented lid vessels performed better showing faster growth and heathier plants with many leaves when compared with that of the sealed vessels. The results of photoautotrophic growth processes carried out in vented vs sealed growth chambers is shown in FIG. 7.

### Example 4: Generation of an In-Vitro Clone Mother

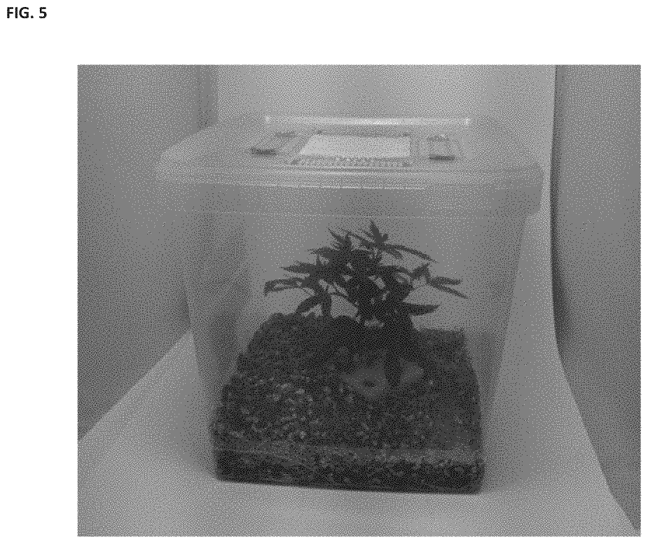

The acclimatized plants from Example 3 are placed in a sterile 5-liter grow vessel as show in FIG. 5 with 300 mL sterile vermiculite substrate and 400 mL of filter-sterilized low-strength hydroponics media with a vented lid of a diameter of 3.175 centimeters comprising 0.2- micron pore size filters. A sterile container ranging in size from 0.5 liter to 100 L with adequate ventilation can also be used, and any sterile growth medium such as sponge cube, rockwool, or perlite can be used for the growth of the clone mother plant. The grow vessel as seen in FIG. 5 is then placed under light conditions with an intensity of 60-200 μmol/m⁻²/s⁻¹ and allowed to mature to a sufficient size and then the clone mother can be used for harvesting cut sections as in Example 4.

### Example 5: Production and Use of Vented Lid for Photoautotrophic Propagation

In a heterotrophic plant tissue culture, the sucrose present in the gel media serves as a carbon source for the plants to metabolize through cellular respiration. This sucrose source is not available for plants grown under photoautotrophic conditions since the gel media used for photoautotrophic growth lacks sucrose. Hence the photoautotrophic plant tissue culture must use carbon dioxide from the air as the carbon source since no sucrose is provided to the plant. Although ventilation is often used in plant tissue culture, the primary function of such ventilation is gaseous exchange; allowing the evacuation active or passive exchange of ethylene, carbon dioxide and other gases between the culture vessel and culture environment to mitigate symptoms/side effects associated with micropropagation. Vented lids known in the art and used for plant growth have membranes for gas exchange that are made from uniform extrusions of different plastic resins and do not allow water vapor to diffuse across them. Such vented lids are not suitable for plants or plant culture under photoautotrophic conditions.

The vented lid allows for the optimum exchange of gases such as carbon dioxide, ethylene, etc., and water vapor from the surrounding ambient air. This example demonstrates the production and the use of a vented lid in the growth of plants under photoautotrophic conditions. The vented lid also prevents the entry of pathogenic microorganisms into the growth vessel, thereby preventing a bacterial or viral attack on the plants inside the growth vessels. The net export of water vapor out of the growth vessel reduces humidity and induces a higher vapor pressure deficit thereby accelerating transpiration and metabolism. The vented lid thus facilitates faster growth of plants inside the vessel and protects the plants from many of the negative symptoms associated with plant tissue culture such as hyperhydricity and chlorosis.

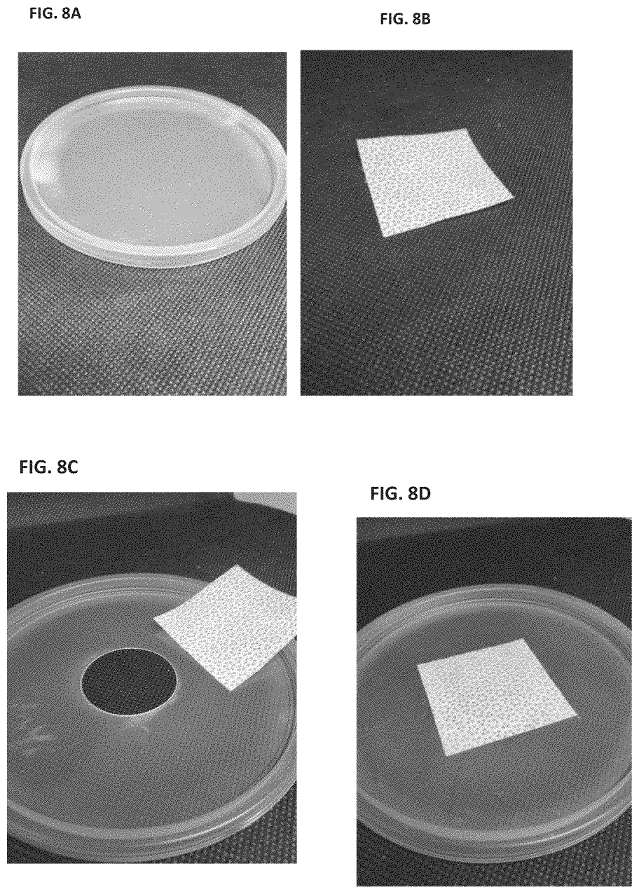

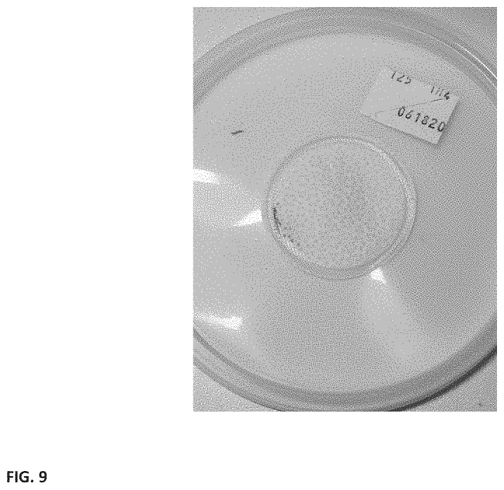

The vented lids are manufactured by laser cutting a circular hole or square opening in the plastic closure. A gas and moisture permeable membrane made from spun bound or non-woven polypropylene fabric is then affixed over the aperture in a continuous seal on the surface of the lid with heat-resistant adhesive, direct heat or by ultrasonic welding. The pores in the polypropylene fabric range from 0.01 to 0.2 microns which facilitate exchange of gases and water vapor but not pathogens. The size of pores in the fabric can be 0.01 microns, 0.02 microns, 0.03 microns, 0.04 microns, 0.05 microns, 0.06 microns, 0.07 microns, 0.08 microns, 0.09 microns, 0.1 microns, 0.12 microns, 0.14 microns, 0.16 microns, 0.18 microns and 0.2 microns. The size of the aperture on the closure and membrane is significantly larger than typical vents-measuring between 2.5 cm to 7.5 cm. In some aspects the aperture can be 2.5 cm, 3 cm, 3.5 cm, 4 cm, 4.5 cm, 5 cm, 5.5 cm, 6 cm, 6.5 cm, 7 cm and 7.5 cm. The diameter of the lids varies from 5 cm to 50 cm, in some aspects, the diameter of the lids can be 5 cm, 10 cm, 15 cm, 20 cm, 25 cm, 25 cm, 30 cm, 35 cm, 40 cm, 45 cm and 50 cm. Representative examples of vented lids are shown in FIGS. 8 and 9. The vented lids are made from materials such as polypropylene, polyethylene, poly ethylene terephthalate, poly vinyl chloride, polycarbonate, polystyrene, and glass. The membrane or fabric covering the aperture of the vented lid is made from polypropylene, cellulose, and nylon which have pore sizes ranging from 0.01-0.2 microns.

The lids thus produced can be used with plants that are at any stage of plant growth under photoautrophic conditions. Typically, as plants mature, the size of lid is also increased to facilitate greater exchange of gases and water vapor which in turn promotes the growth of mature plants.

### OTHER EMBODIMENTS

From the foregoing description, it will be apparent that variations and modifications may be made to the invention described herein to adopt it to various usages and conditions, including the use of different strains of Cannabis, different plant varieties, variations in temperature, nutrient solutions, grow vessels, sugar concentrations, vent size, and light flux, are within the scope according to the invention. Other embodiments according to the invention are within the following claims.

Recitation of a listing of elements in any definition of a variable herein includes definitions of that variable as any single element or combination (or sub combination) of listed elements. Recitation of an embodiment herein includes that embodiment as any single embodiment or in combination with any other embodiments or portions thereof.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Other embodiments are within the following claims.

1. A method of in vitro propagation of plant tissue, said method comprising:
a. placing a first node segment of a plant in a low sugar gel media contained in a semi permeable vessel,
b. exposing the vessel to a light environment,
c. permitting passive diffusion of gases from the vessel for a time sufficient to facilitate photosynthetic growth of the first node segment, said photosynthetic growth resulting in the propagation of the first node segment into a propagule comprising one or more node segments.

2. The method of claim 1, wherein the low sugar gel media comprises less than 5 g of soluble carbohydrates.

3. The method of claim 1, wherein the low sugar gel media comprises soluble carbohydrates selected from the group consisting of sucrose, glucose, fructose, galactose, and maltose.

4. The method of claim 1, wherein the light environment is a low light environment.

5. The method of claim 4, wherein the light environment has a flux of less than 100 μmol/m⁻²/s⁻¹.

6. The method of claim 1, wherein the time sufficient is 10-40 days.

7. The method of claim 1, wherein the first node segment is at least 1 cm in length.

8. The method of claim 1, wherein the first node segment is obtained by dissecting stem sections of a sterile clone mother plant.

9. The method of claim 1 further comprising the step of dissecting said one or more node segments to generate a plurality of node segments and repeating steps (a) to (c).

10. The method of claim 1, wherein the vessel comprises a vented lid which permits said passive diffusion of gases for photosynthetic growth.

11. The method of claim 1, wherein the vented lid comprises a pore size of 0.2 microns.

12. The method of claim 1, wherein the plant is selected from the group consisting of Cannabis sativa, Cannabis indica, and Cannabis ruderalis.

13. The method of claim 1, wherein the propagule comprises one or more fibrous roots.

14. The method of claim 1, wherein the low sugar gel media contains a cytokinin and/or an auxin.

15. A method of in vitro multiplication of plant tissue, said method comprising:
a. placing a propagule of claim 1 in a solid substrate or aggregate augmented with a liquid nutrient solution contained in a semi permeable vessel,
b. exposing the vessel to a light environment,
c. permitting a passive diffusion of gases from the vessel for a time sufficient to facilitate photosynthetic growth of the first node segment, said photosynthetic growth resulting in the propagation of the first node segment into one or more node segments.

16. The method of claim 15, wherein the light environment is a moderate light environment.

17. The method of claim 16, wherein the moderate light environment has a flux of less than 66 μmol/m⁻²/s⁻¹.

18. The method of claim 15, wherein the time sufficient is 7-10 days.

19. The method of claim 15, wherein the vessel further comprises a lid.

20. The method of claim 19, wherein the lid is a vented lid.

21. The method of claim 20, wherein the vented lid prevents the entry of pathogenic microbes into the vessel.

22. The method of claim 20, wherein the vented lid facilitates gaseous exchange and water vapor between the vessel and its surrounding environment.

23. The method of claim 20, wherein the vented lid comprises spun bound or non-woven polypropylene membrane or fabric.

24. The method of claim 23, wherein the fabric has a pore size ranging from 0.01-0.2 microns.

25. A lid for attachment to a growth vessel having an open top, wherein said growth vessel is suitable for photoautrophic plant growth and said lid configured to seal said open top of said growth vessel, wherein said lid comprises an aperture covered by a membrane capable of permitting free exchange of gases and water vapor into and out of the growth vessel, and wherein said membrane prevents the entry of microbial pathogens into said growth vessel, wherein the size of aperture ranges from 2.5 cm to 7 cm.

26. The lid of claim 25, wherein the membrane has a pore size ranging from 0.01-0.2 microns.

27. The lid of claim 25, wherein the diameter of lid ranges from 5 to 50 cm.

28. The lid of claim 25, wherein said lid is made from materials selected from the group consisting of polypropylene, polyethylene, polyethylene terephthalate, poly vinyl chloride, polycarbonate, polystyrene, and glass.

29. The lid of claim 25, wherein the membrane is made from material selected from polypropylene, cellulose, and nylon.
